(19) United States
(12) Patent Application Publication
(10) Pub. No.: US 2003/0159221 A1
(43) Pub. Date: Aug. 28, 2003

(54) Oxidation dyeing composition for keratinous fibres with a particular paraphenylene-diamine derivative and a particular direct dyeing agent

(76) Inventor: Gerard Lang, Saint Prix (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/333,663

(22) PCT Filed: Mar. 5, 2001
(86) PCT No.: PCT/FR01/00644

(30) Foreign Application Priority Data
Mar. 6, 2000 (FR) 00/02862

(51) Int. Cl. A61K 7/13

(52) U.S. Cl. 8/405; 8/406; 8/408; 8/415; 8/421

(57) ABSTRACT

The invention concerns an oxidation dyeing composition for keratinous fibres, and in particular human keratinous fibres such as hair comprising, in a medium suitable for dyeing, at least an oxidation base selected among certain substituted para-phenylenediamine derivatives and their addition salts with an acid, and at least a synthetic direct dyeing agent selected among the azo, quinoid, triarylmethane, indoamino, azine dyes and/or a natural dye. The invention also concerns a dyeing method using said composition.

The invention relates to a composition for the oxidation dyeing of keratin fibers, and in particular of human keratin fibers such as the hair, comprising, in a medium that is suitable for dyeing, at least one oxidation base chosen from certain substituted para-phenylenediamine derivatives and the addition salts thereof with an acid and at least one particular synthetic direct dye and/or a natural dye, and also to the dyeing process using this composition.

It is known practice to dye keratin fibers, and in particular human hair, with dye compositions containing oxidation dye precursors, in particular ortho- or para-phenylenediamines, ortho- or para-aminophenols, and heterocyclic bases, which are generally known as oxidation bases. These oxidation dye precursors (oxidation bases) are colorless or weakly colored compounds which, when combined with oxidizing products, can give rise to colored compounds and dyes by a process of oxidative condensation.

It is also known that the shades obtained with these oxidation bases can be varied by combining them with couplers or coloration modifiers, the latter being chosen in particular from aromatic meta-diamines, meta-aminophenols, meta-diphenols and certain heterocyclic compounds.

The variety of molecules used as oxidation bases and couplers allows a wide range of colors to be obtained.

The so-called “permanent” coloration obtained by means of these oxidation dyes should moreover satisfy a certain number of requirements. Thus, it should allow shades to be obtained in the desired intensity and it should have good staying power with respect to external agents (light, bad weather, washing, permanent-waving, perspiration or rubbing).

The dyes should also allow gray hair to be covered and, finally, they should be as unselective as possible, i.e. they should allow only the smallest possible differences in coloration to be obtained along the same keratin fiber, which may indeed be differently sensitized (i.e. damaged) between its end and its root.

It has already been proposed, especially in patent applications JP-11-158 046, JP-11-158 047 and JP-11-158 048 to use compositions for the oxidation dyeing of keratin fibers, containing substituted para-phenylenediamine derivatives as oxidation dye precursors. However, the colorations obtained by using these compositions are not always sufficiently intense, chromatic or resistant to the various attacking factors to which the hair may be subjected.

However, the Applicant has now discovered that it is possible to obtain novel oxidation dyes, which are capable of giving chromatic, intense, attractive colorations in varied shades, which are relatively unselective and resistant to the various attacking factors to which the fibers may be subjected, by combining at least one oxidation base chosen from certain para-phenylenediamine derivatives of formula (I) defined below and the addition salts thereof with an acid, and at least one particular direct dye and/or a natural dye.

This discovery forms the basis of the present invention.

Thus, a first subject of the invention is a composition for the oxidation dyeing of keratin fibers, in particular of human keratin fibers such as the hair, characterized in that it comprises, in a medium that is suitable for dyeing:

(A) at least one oxidation base chosen from the substituted para-phenylenediamine derivatives of formula (I) below, and the addition salts thereof with an acid:

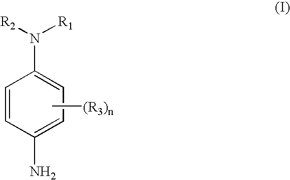

in which:

R₁ and R₂ can take one of the following meanings i) to v) below:

i) R₁ and R₂ simultaneously represent a —(CH₂)₂CHOHCH₂OH radical; or

ii) R₁ represents a —CH₂(CHOH)₄CH₂OH radical and R₂ represents a hydrogen atom, an alkyl or aryl radical or a heterocycle; or

iii) R₁ represents an alkyl or aryl radical or a heterocycle and R₂ represents an alkylene radical —(CH₂)ₘ— in which m is an integer equal to 2 or 3, said alkylene radical forming a ring together with the nitrogen atom, the carbon atom of the benzene ring bearing the nitrogen atom and one of the two carbon atoms of the benzene ring that are adjacent to it, it being understood that when R₁ is an alkyl or aryl radical, then either R₁ or said alkylene radical is substituted with a radical containing at least one nitrogen, oxygen or sulfur atom;

iv) R₁ represents a radical —(CH₂CH₂O)ₚR₄ in which p is an integer between 2 and 8 inclusive, R₄ and R₂, which may be identical or different, represent a hydrogen atom, an alkyl or aryl radical or a heterocycle;

v) R₁ and R₂ form, together with the nitrogen atom to which they are attached, a 5-, 6- or 7-membered saturated heterocycle, said heterocycle being substituted with at least one radical containing at least one carbon, nitrogen, oxygen or sulfur atom;

R₃ represents a halogen atom, an alkyl or aryl radical, a cyano, nitro, hydroxyl, carboxyl, sulfo, alkoxy, aryloxy, cyanoamino, amino, anilino, ureido, sulfamylamino, mono- or dialkylsulfamylamino, alkylthio, arylthio, alkoxycarbonylamino, sulfonamido, carbamyl, mono- or dialkylcarbamylsulfamyl, sulfonyl, alkoxycarbonyl, azo, acyloxy, carbamyloxy, mono- or dialkylcarbamyloxy, silyl, silyloxy, aryloxycarbonylamino, imido, sulfinyl, phosphonyl, aryloxycarbonyl, acyl or mercapto radical, a heterocycle or a heterocycle linked to the benzene ring of formula (I) via an ether or thio bond;

said alkyl radicals containing from 1 to 25 carbon atoms and possibly being linear, branched or cyclic and possibly being substituted with one or more radicals, and in this case possibly representing a mono- or polyhydroxyalkyl radical, an alkoxyalkyl radical, an aminoalkyl radical optionally substituted on the nitrogen atom, or a carboxyalkyl, alkylcarboxyalkyl, thioalkyl, alkylthioalkyl, cyanoalkyl, trifluoroalkyl, sulfoalkyl, phosphoalkyl or haloalkyl radical;

said alkoxy radicals containing from 1 to 25 carbon atoms and possibly being linear, branched or cyclic; said aryl radicals containing from 6 to 26 carbon atoms and possibly being substituted with one or more radicals chosen from alkyl, substituted alkyl and alkoxy radicals;

the heterocycles being monocyclic or polycyclic, each ring being 3-, 4-, 5- or 6-membered and possibly containing one or more hetero atoms, it being understood that in the case of polycyclic heterocycles, at least one of the rings contains at least one hetero atom such as N, O or S;

n is an integer between 0 and 4; it being understood that when n is greater than 1, then the radicals R₃ may be identical or different and may together form a 3-, 4-, 5- or 6-membered saturated or unsaturated ring; with the proviso that:

1) when R₁ and R₂ have the meanings defined in point v), then the compounds of formula (I) do not contain more than 3 hydroxyl radicals;

2) when R₁ and R₂ have the meanings defined in point v) and when R₁ and R₂ form a pyrrolidine ring substituted with a carbamoyl radical on the carbon in the position alpha to the nitrogen atom to which they are attached, then n is other than 0; or the pyrrolidine ring bears at least two substituents;

3) when R₁ and R₂ have the meanings defined in point v) and when R₁ and R₂ form a pyrrolidine ring substituted with a hydroxymethyl radical on the carbon in the alpha position relative to the nitrogen atom to which they are attached and when n=0 or 1, then either said ring bears at least two additional substituents, or said ring bears only one second substituent other than a hydroxyl radical on the carbon located in the position relative to the nitrogen atom and relative to the carbon bearing said hydroxymethyl substituent; or alternatively when R₁ and R₂ have the meanings defined in point v) and when R₁ and R₂ form a pyrrolidine ring substituted with a hydroxymethyl radical on the carbon located in the alpha position relative to the nitrogen atom to which they are attached, and when n=1, then R₃ is other than an alkyl or mono- or polyhydroxyalkyl radical;

4) when R₁ and R₂ have the meanings defined in point iii), the compounds of formula (I) must satisfy at least one of the following four conditions:

a) irrespective of the value of n, the alkylene ring formed by the radical R₂ comprises a substituent in addition to the radical R₁; or

b) n is greater than 1; or

c) when n is equal to 1, then R₃ represents an aryl radical or a heterocycle; or

d) when n is equal to 0 or 1, then R₁ represents an aryl radical, a heterocycle or a substituted alkyl radical other than a monohydroxyalkyl radical;

5) R₁ and R₂ form a ring other than piperazine or diazacycloheptane;

(B) at least one synthetic direct dye chosen from azo dyes, quinone dyes, triarylmethane dyes, indoamine dyes or azine dyes and/or a natural dye.

The addition salts with an acid of the substituted para-phenylenediamine derivatives of formula (I) that may be used in the dye compositions according to the invention are chosen especially from the hydrochlorides, hydrobromides, sulfates, tartrates, lactates and acetates.

The dye composition in accordance with the invention thus defined gives, after mixing with an oxidizing composition, chromatic, intense, attractive colorations in varied shades, with low selectivity and excellent resistance properties not only with respect to atmospheric agents such as light and bad weather, but also with respect to perspiration and the various treatments to which the hair may be subjected.

Another subject of the invention relates to a ready-to-use composition for the oxidation dyeing of keratin fibers, which comprises, in a medium that is suitable for dyeing, at least one substituted para-phenylenediamine derivative of formula (I), at least one synthetic azo, quinone, triarylmethane, indoamine or azine direct dye and/or a natural dye, and at least one oxidizing agent.

For the purposes of the present invention, the expression “ready-to-use composition” means any composition intended to be applied immediately to keratin fibers.

The invention also relates to a process for the oxidation dyeing of keratin fibers, and in particular of human keratin fibers such as the hair, which consists in applying to the fibers a dye composition containing, in a medium that is suitable for dyeing, at least one substituted para-phenylenediamine derivative of formula (I) in combination with at least one synthetic azo, quinone, triarylmethane, indoamine or azine direct dye and/or a natural dye, the color being developed at alkaline, neutral or acidic pH with the aid of a composition containing at least one oxidizing agent, which is mixed just at the time of use to the dye composition, or which is applied sequentially without intermediate rinsing.

A subject of the invention is also a multi-compartment dyeing device or “kit” for the oxidation dyeing of keratin fibers, in particular of human keratin fibers such as the hair, which comprises at least one compartment containing at least one substituted para-phenylenediamine derivative of formula (I) and at least one synthetic azo, quinone, triarylmethane, indoamine or azine direct dye and/or a natural dye, and at least one other compartment containing at least one oxidizing agent.

Another multi-compartment dyeing device comprises at least one compartment containing at least one substituted para-phenylenediamine derivative of formula (I), at least one compartment containing at least one synthetic azo, quinone, triarylmethane, indoamine or azine direct dye and/or a natural dye, and at least one other compartment containing at least one oxidizing agent

However, other characteristics, aspects, subjects and advantages of the invention will emerge even more clearly on reading the description and the examples that follow.

According to the present invention, the substituted para-phenylenediamine derivatives that are preferably used are those of formula (I) above in which:

R₁ and R₂ can take one of the meanings i) to v) below:

i) R₁ and R₂ simultaneously represent a —(CH₂)₂CHOHCH₂OH radical; or

ii) R₁ represents a —CH₂(CHOH)₄CH₂OH radical and R₂ represents a hydrogen atom or an alkyl radical; or

iv) R₁ represents a radical —(CH₂CH₂O)ₚR₄ in which p is an integer between 2 and 8 inclusive, R₄ and R₂, which may be identical or different, represent a hydrogen atom or an alkyl radical;

v) R₁ and R₂ form, together with the nitrogen atom to which they are attached, a 5-, 6- or 7-membered saturated heterocycle, said heterocycle being substituted with at least one radical containing at least one carbon, nitrogen or oxygen atom, which is not in a meta position relative to the nitrogen atom of the heterocycle;

R₃ represents a halogen atom, an alkyl or aryl radical or a heterocycle,

n is an integer equal to 0, 1 or 2.

More particularly, according to the invention, in formula (I), R₁ and R₂ form a pyrrolidine heterocycle.

Among the substituted para-phenylenediamine derivatives of formula (I) above that may be mentioned most particularly are 1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis-(3′,4′-dihydroxybutyl)-3-propyl-para-phenylenediamine, 1-N,N-bis(3′,4′-dihydroxybutyl)-3-methoxy-para-phenylenediamine, 1-N,N-bis(3′,4′-dihydroxybutyl)-3-ethoxy-para-phenylenediamine, 1-N,N-bis(3′,4′-dihydroxybutyl)-3-propyloxy-para-phenylenediamine, 1-N,N-bis(3′,4′-dihydroxybutyl)-3-hexyloxy-para-phenylenediamine, 1-N,N-bis(3′,4′-dihydroxybutyl)-3-(1″-N-3″,5″-dimethylpyrazolyl-para-phenylenediamine, 1-N,N-bis(3′,4′-dihydroxybutyl)-3-ureido-para-phenylenediamine, 1-N,N-bis(3′,4′-dihydroxybutyl)-3-trimethyl-1″,3″,3″-ureido-para-phenylenediamine, 1-N,N-bis(3′,4′-dihydroxybutyl)-3-dimethylamino-para-phenylenediamine, 1-N,N-bis(3′,4′-dihydroxybutyl)-3-methylthio-para-phenylenediamine, 1-N,N-bis(3′,4′-dihydroxybutyl)-3-ethylthio-para-phenylenediamine, 1-N,N-bis(3′,4′-dihydroxybutyl)-3-mercapto-para-phenylenediamine, 1-N,N-bis(3′,4′-dihydroxybutyl)-3-n-butylthio-para-phenylenediamine, 1-N,N-bis(3′,4′-dihydroxybutyl)-3-n-octylthio-para-phenylenediamine, 1-N,N-bis(3′,4′-dihydroxybutyl)-3-mercaptoethyl-para-phenylenediamine, 1-N,N-bis(3′,4′-dihydroxybutyl)-3-mercaptoethylthio-para-phenylenediamine, 1-N,N-bis(3′,4′-dihydroxybutyl)-3-β-hydroxyethylthio-para-phenylenediamine, 1-N-(2′,3′,4′,5′,6′-pentahydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(2′,3′,4′,5′,6′-pentahydroxyhexyl)-3-methoxy-para-phenylenediamine, 1-N-(2′,3′,4′,5′,6′-pentahydroxyhexyl)-1-N-(4″-N″-methylpiperidyl)-3-ethoxy-para-phenylenediamine, 1-N-(2′,3′,4′,5′,6′-pentahydroxyhexyl)-3-isopropyloxy-para-phenylenediamine, 1-N-(2′,3′,4′,5′,6′-pentahydroxyhexyl)-3-dimethylamino-para-phenylenediamine, 1-N-(2′,3′,4′,5′,6′-pentahydroxyhexyl)-3-methylthio-para-phenylenediamine, 1-N-(2′,3′,4′,5′,6′-pentahydroxyhexyl)-3-mercapto-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-isooctyloxy-para-phenylenediamine, 1-N-(methyl)-1-N-(2′,3′,4′,5′,6′-pentahydroxyhexyl)-3-isopropyloxy-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-3-hydroxyethyloxy-para-phenylenediamine, 1-N-.(methyl)-1-N-(2′,3′,4′,5′,6′-pentahydroxyhexyl)-3-mercaptoethyloxy-para-phenylenediamine, 1-N-(methyl)-1-N-(2′,3′,4′,5′,6′-pentahydroxyhexyl)-para-phenylenediamine, 1-N-(phenyl)-1-N-(2′,3′,4′,5′,6′-pentahydroxyhexyl)-3-ethyloxy-para-phenylenediamine, 1-N-(4″-N-methylpiperidyl)-1-N-(2′,3′,4′,5′,6′-pentahydroxyhexyl)-3-ethyloxy-para-phenylenediamine, 4-N-(methyl)-4-N-(2′,3′,4′,5′,6′-pentahydroxyhexyl)-amino-7-amino-1-methylindole and 1-N-(hydroxyethyloxyethyl)-1-N-(2′,3′,4′,5′,6′-pentahydroxyhexyl)-3-ethyl-para-phenylenediamine, and the addition salts thereof with an acid, and among which, the ones that are even more particularly preferred are: 1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis(3′,4′-dihydroxybutyl)-3-propyl-para-phenylenediamine, 1-N-(2′,3′,4′,5′,6′-pentahydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-para-phenylenediamine and 1-N-(hydroxyethyloxyethyl)-1-N-(2′,3′,4′,5′,6′-pentahydroxyhexyl)-3-ethyl-para-phenylenediamine, and the addition salts thereof with an acid.

Mention may also be made most particularly of 1-N-(3′,4′-dihydroxybutyl)-5-aminoindoline, 1-(2′-hydroxyethyl)-2-methyl-5-aminoindoline, 1-methyl-2-hydroxymethyl-5-aminoindoline, 6-methyl-2-hydroxyethyl-5-aminoindoline, 2-hydroxyethyloxyethyl-5-aminoindoline, 2-hydroxyethyloxyethyloxyethyloxyethyl-5-aminoindoline, 2-hydroxyethyloxyethyloxyethyloxy-ethyloxyethyloxyethyl-6-isopropyl-5-aminoindoline, 2-hydroxyethyl-3-methyl-5-aminoindoline, 2-hydroxyethyloxyethyloxyethyl)-5-aminoindoline, 1-carboxymethyl-2,3,3-trimethyl-5-aminoindoline, 1-methylsulfonamidoethyl-3-methyl-5-aminoindoline, 1-ureidoethyl-6-methoxy-5-aminoindoline, 1-(2′,3′,4′,5′,6′-pentahydroxyhexyl)-5-aminoindoline, 1-N-(2′-mercaptoethyl)-5-aminoindoline, the dimethyl ester 6-amino-1-methyl-1,2,3,4-tetrahydrofuro-[2,3,h]-quinoline 4-methyl ester of phosphoric acid, 6-amino-1,2,2-trimethyl-4-trimethylsilanyloxy-1,2,3,4-tetrahydroquinoline, 6-amino-1-hexyl-2,2,7-trimethyl-4-mercaptomethyl-1,2,3,4-tetrahydroquinoline, 6-amino-1-(3′,4′-dihydroxybutyl)-2,2,3-trimethyl-1,2,3,4-tetrahydroquinoline, 6-amino-1-(ethoxyethoxy-ethoxyethoxy-3′,4′-dihydroxybutyl)-2,2,3,7-tetramethyl-1,2,3,4-tetrahydroquinoline, 6-amino-1-(hydroxyethyloxyethyloxyethyloxyethyl)-2,2,3-trimethyl-1,2,3,4-tetrahydroquinoline, 6-amino-1-(hydroxyethyloxyethyl)-2,2,3-trimethyl-1,2,3,4-tetrahydroquinoline, 6-amino-1-(ethylbis-(hydroxyethyloxyethyloxyethyloxyethyl))-2,2,3,7-tetramethyl-1,2,3,4-tetrahydroquinoline, 1-(carboxymethyl)-2,2,3,7-tetramethyl-1,2,3,4-tetrahydroquinoline, 1-(hydroxypropyl)-2,2,3-trimethyl-7-methoxy-1,2,3,4-tetrahydroquinoline, 6-amino-1-(hydroxyethyloxyethyloxyethyl)-2,2,3-trimethyl-7-isopropyl-1,2,3,4-tetrahydroquinoline, 6-amino-1-(hydroxyethyloxyethyloxyethyloxyethyloxyethyloxyethyl-oxyethyloxyethyl)-2,2,3-trimethyl-7-isopropyl-1,2,3,4-tetrahydroquinoline, 6-amino-1-(hydroxyethyloxyethyloxyethyloxyethyloxyethyloxyethyl-oxyethyloxyethyl)-2,2,3-trimethyl-1,2,3,4-tetrahydroquinoline, 6-amino-1-(mercaptoethyl)-1,2,3,4-tetrahydroquinoline, 6-amino-1-(3′,4′-dihydroxybutyl)-2,2,3-trimethyl-7-isopropyl-1,2,3,4-tetrahydroquinoline, 6-amino-1-(3′,4′-dihydroxybutyl)-2,2,7-trimethyl-4-hydroxymethyl-1,2,3,4-tetrahydroquinoline, 6-amino-1-(3′,4′-dihydroxybutyl)-2,2-dimethyl-4-hydroxymethyl-7-isopropyl-1,2,3,4-tetrahydroquinoline, 6-amino-1-(3-hydroxypropyl)-2,2-dimethyl-4-hydroxymethyl-7-isopropyl-1,2,3,4-tetrahydroquinoline, 6-amino-1-(hydroxyethyloxyethyloxyethyloxyethyloxyethyloxy-ethyloxyethyloxyethyl)-2,2-dimethyl-4-hydroxymethyl-7-isopropyl-1,2,3,4-tetrahydroquinoline, 6-amino-1-(hydroxyethyloxyethyloxyethyloxyethyl)-2,2-dimethyl-4-hydroxymethyl-7-isopropyl-1,2,3,4-tetrahydroquinoline, 6-amino-1-(hydroxyethyloxyethyloxyethyloxyethyloxy-ethyl)-2,2-dimethyl-7-isopropyl-1,2,3,4-tetrahydroquinoline, 6-amino-1-(hydroxyethyloxyethyloxyethyloxyethyloxyethyloxyethyl)-2,2-dimethyl-7-isopropyl-1,2,3,4-tetrahydroquinoline, 6-amino-1-(hydroxyethyloxyethyl)-2,2-dimethyl-1,2,3,4-tetrahydroquinoline, 6-amino-1,2,2,4,7-pentamethyl-3-hydroxy-1,2,3,4-tetrahydroquinoline, 6-amino-1-(3′-hydroxypropyl)-4-(hydroxyethyloxyethyloxyethyloxy-ethyl)-2,2-dimethyl-7-isopropyl-1,2,3,4-tetrahydroquinoline, 6-amino-1-(hydroxyethyloxyethyloxyethyloxyethyl)-4,4-dimethyl-1,2,3,4-tetrahydroquinoline, 6-amino-1-(3′,4′-dihydroxybutyl)-2,2-dimethyl-7-isopropyl-1,2,3,4-tetrahydroquinoline, 6-amino-1-(hydroxyethyloxyethyloxyethyloxyethyl)-4-(hydroxyethyloxyethyloxyethyloxyethyl)-2,2,7-trimethyl-1,2,3,4-tetrahydroquinoline, 6-amino-1-(hydroxyethyloxyethyloxyethyloxyethyl)-2,2-dimethyl-7-isopropyl-1,2,3,4-tetrahydroquinoline, 6-amino-1-(2′,3′,4′,5′,6′-pentahydroxyhexyl)-2,2,4-trimethyl-7-isopropyl-1,2,3,4-tetrahydroquinoline, 6-amino-1-(mercaptoethyl)-2,2,4-trimethyl-7-(2′,3′-dihydroxypropyloxy)-1,2,3,4-tetrahydroquinoline, 6-amino-1-(3′,4′-dihydroxybutyl)-2,2,7-trimethyl-3-mercaptomethyl-1,2,3,4-tetrahydroquinoline, 6-amino-1-(ureidoethyl)-2,2,4-trimethyl-1,2,3,4-tetrahydroquinoline, 6-amino-2,2-dimethyl-7-chloro-1,2,3,4-tetrahydroquinoline-1-propylsulfonic acid, 6-amino-1-(4′-pyridinyl)-2,2,7-trimethyl-1,2,3,4-tetrahydroquinoline, 6-amino-1-(3′,4′-dihydroxybutyl)-2,2,4,7-tetramethyl-1,2,3,4-tetrahydroquinoline, 6-amino-1,7-diisopropyl-2,2-dimethyl-4-trimethylsilanyloxy-1,2,3,4-tetrahydroquinoline, 6-amino-1,2,2,4-tetramethyl-3-hydroxy-1,2,3,4-tetrahydroquinoline and 6-amino-1-bromo-2,2-dimethyl-4-mercapto-7-isopropyloxy-1,2,3,4-tetrahydroquinoline, and the addition salts thereof with an acid.

Mention may also be made most particularly of 1-(4′-amino-3′-isopropyloxyphenyl)-2,6-dimethylpyrrolidine, 1-(4′-amino-3′-methylphenyl)-3-hydroxyethyloxypyrrolidine, 1-(4′-amino-3′-methylphenyl)-4-hydroxy-2-methylpyrrolidine, 1-(4′-amino-3′-methylphenyl)-3-methylsulfonamidopyrrolidine, 1-(4′-amino-3′-phenoxyphenyl)-3-methylsulfonamidopyrrolidine, 3-n-butylpyrrolidine-1-(4′-amino-3′-phenylsulfonic acid), 1-(4′-amino-3′-acetylaminophenyl)-3-hydroxymethylpyrrolidine, 7-amino-4-(2′-methyl)-pyrrolidinylbenzofuran, 1-(4′-aminophenyl)-2-(4″-aminophenoxymethyl)piperidine, 1-(4′-amino-3′-acetylphenyl)-4-hydroxypiperidine, 1-(4′-aminophenyl)-2-(hydroxyethyl)piperidine, 1-(4′-amino-3′-methoxyphenyl)-2,6-dihydroxymethylpiperidine, 1-(4′-amino-3′-isopropyloxyphenyl)-2,6-dimethylpiperidine, 1-(4′-amino-3′-isopropylphenyl)-2-hydroxymethylpiperidine, 1-(4′-amino-3′-isopropyloxyphenyl)-2-hydroxymethylpiperidine, 1-(4′-amino-3′-aminophenyl)-2-hydroxymethylpiperidine, 1-(4′-amino-3′-dimethylaminophenyl)-2-mercaptoethyloxyethylpiperidine, 1-(4′-amino-3′-(2″′,4″′-dichloro)anilinophenyl)-4-methylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-(4′-amino-3′-sulfamoylaminophenyl)-2,7-dimethylazacycloheptane, 1-(4′-amino-3′-methylthiophenyl)-2,7-dimethylazacycloheptane, 1-N-4′-hydroxybutyl-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N-phenyl-1-N-(hydroxyethyloxyethyl)-para-phenylenediamine, 1-N-benzyl-1-N-(hydroxyethyloxyethyloxyethyloxyethyloxyethyl)-3-trimethylsilyl-para-phenylenediamine, 1-N-methyl-1-N-(hydroxyethyloxyethyloxyethyloxyethyloxyethyloxy-ethyloxyethyloxyethyl)-3-trimethylsilyloxy-para-phenylenediamine, 1-N-ethyl-1-N-(methoxyethyloxyethyloxyethyloxyethyloxyethyl)-3-phenoxycarbonylamino-para-phenylenediamine, 1-N-methyl-1-N-(methoxyethyloxyethyloxyethyl)-3-(2′,5′-dioxopyrrolidinyl)-para-phenylenediamine, 1-N-ethyl-1-N-(hydroxyethyloxyethyloxyethyl)-3-4′-pyridinylthio-para-phenylenediamine, 1-N-propyl-1-N-(hydroxyethyloxyethyloxyethyl)-3-sulfinyl-para-phenylenediamine, 1-N-methyl-1-N-(hydroxyethyloxyethyl)-3-phenoxycarbonyl-para-phenylenediamine, 1-N,N-bis(hydroxyethyloxyethyloxyethyloxyethyl)-para-phenylenediamine, 1-N,N-bis(methoxyethyloxy-ethyloxyethyloxyethyl)-3-isopropyloxy-para-phenylenediamine, 1-N,N-bis(hydroxyethyloxy-ethyloxyethyl)-3-isopropyloxy-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis(methoxyethyloxyethyl-oxyethyloxyethyloxyethyloxyethyloxyethyl)-3-methoxy-para-phenylenediamine, 1-N,N-bis(hydroxyethyloxy-ethyloxyethyloxyethyloxyethyloxyethyloxyethyloxyethyl)-3-methyl-para-phenylenediamine, 1,N,N-bis(hydroxyethyloxyethyloxyethyloxyethyl)-3-isopropyloxy-para-phenylenediamine, 1-N,N-bis(hydroxyethyloxyethyl)-3-mercaptoethyl-para-phenylenediamine and 1-N,N-bis(benzyloxyethyloxyethyloxyethyl)-3-isopropyl-para-phenylenediamine, and the addition salts thereof with an acid,

and among which, the ones that are even more particularly preferred are:

1-(4′-amino-3′-methylphenyl)-3-hydroxyethyloxypyrrolidine, 1-(4′-amino-3′-methylphenyl)-4-hydroxy-2-methylpyrrolidine, 1-(4′-amino-3′-methylphenyl)-3-methylsulfonamidopyrollidine, 1-(4′-amino-3′-phenoxyphenyl)-3-methylsulfonamidopyrrolidine, 1-(4′-aminophenyl)-2-(4″-aminophenoxymethyl)piperidine, 1-(4′-aminophenyl)-2-hydroxyethyl)piperidine, 1-(4′-amino-3′-isopropylphenyl)-2-hydroxymethylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-N-4′-hydroxybutyl-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N,N-bis(hydroxyethyloxyethyloxyethyloxyethyl)-para-phenylenediamine, 1-N,N-bis(hydroxyethyloxy-ethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyloxyethyloxy-ethyloxyethyloxyethyl)-3-methyl-para-phenylenediamine and 1-N,N-bis(benzyloxyethyloxyethyloxyethyl)-3-isopropyl-para-phenylenediamine, and the addition salts thereof with an acid.

The para-phenylenediamine derivative(s) of formula (I) used as oxidation base in the dye composition in accordance with the invention is(are) present in concentrations ranging from 0.0001% to 20%, preferably from 0.001% to 15% and even more particularly from 0.01% to 10% by weight relative to the total weight of the composition.

The synthetic direct dyes that may be used according to the invention may be nonionic, anionic or cationic.

Among the synthetic azo direct dyes that may be used according to the invention, mention may be made of the cationic azo dyes described in patent applications WO 95/15144, WO 95/01772 and EP-714954, the content of which forms an integral part of the invention.

Among the azo direct dyes that may also be mentioned are the following dyes, described in the Colour Index International 3rd edition:

Disperse Red 17

Acid Yellow 9

Acid Black 1

Basic Red 22

Basic Red 76

Basic Yellow 57

Basic Brown 16

Acid Yellow 36

Acid Orange 7

Acid Red 33

Acid Red 35

Basic Brown 17

Acid Yellow 23

Acid orange 24

Disperse Black 9.

Among these, mention may also be made of 1-(4′-aminodiphenylazo)-2-methyl-4-bis(β-hydroxyethyl)-aminobenzene and 4-hydroxy-3-(2-methoxyphenylazo)-1-naphthalenesulfonic acid.

Among the synthetic quinone direct dyes that may be mentioned are the following:

Disperse Red 15

Solvent Violet 13

Acid Violet 43

Disperse Violet 1

Disperse Violet 4

Disperse Blue 1

Disperse Violet 8

Disperse Blue 3

Disperse Red 11

Acid Blue 62

Disperse Blue 7

Basic Blue 22

Disperse Violet 15

Basic Blue 99,

and also the compounds:

1-N-methylmorpholiniumpropylamino-4-hydroxyanthraquinone,

1-aminopropylamino-4-methylaminoanthraquinone,

1-aminopropylaminoanthraquinone,

5-β-hydroxyethyl-1,4-diaminoanthraquinone,

2-aminoethylaminoanthraquinone,

1,4-bis(β,γ-dihydroxypropylamino)anthraquinone.

Among the synthetic azine direct dyes that may be mentioned are the following:

Basic Blue 17

Basic Red 2.

Among the synthetic triarylmethane direct dyes that may be used according to the invention, mention may be made of the following:

Basic Green 1

Acid Blue 9

Basic Violet 3

Basic Violet 14

Basic Blue 7

Acid Violet 49

Basic Blue 26

Acid Blue 7

Among the synthetic indoamine direct dyes that may be used according to the invention, mention may be made of the following:

2-β-hydrbxyethylamino-5-[bis(β-4′-hydroxyethyl)-amino]anilino-1,4-benzoquinone

2-β-hydroxyethylamino-5-(2′-methoxy-4′-amino)-anilino-1,4-benzoquinone

3-N-(2′-chloro-4′-hydroxy)phenylacetylamino-6-methoxy-1,4-benzoquinoneimine

3-N-(3′-chloro-4′-methylamino)phenylureido-6-methyl-1,4-benzoquinoneimine

3-[4′-N-(ethylcarbamylmethyl)amino]phenylureido-6-methyl-1,4-benzoquinoneimine.

For the purposes of the invention, the expression “natural dye” means compounds that exist in nature, whether they have been obtained by extraction or reproduced chemically.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin and apigenidin. It is also possible to use extracts or decoctions containing these natural dyes and especially henna-based poultices or extracts.

The synthetic direct dye(s) according to the invention and/or the natural dye(s) represent from 0.001% to 20% by weight approximately relative to the total weight of the composition, and even more preferably from 0.005% to 10% by weight approximately.

Preferably, the compositions of the invention contain at least one coupler.

Among these couplers, mention may be made especially of meta-phenylenediamines, meta-aminophenols, meta-diphenols and heterocyclic couplers such as, for example, indole derivatives, indoline derivatives, benzimidazole derivatives, benzomorpholine derivatives, sesamol derivatives, pyridine, pyrimdine and pyrazole derivatives, and the addition salts thereof with an acid.

These couplers are more particularly chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 2-methyl-1-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one and 1-phenyl-3-methylpyrazol-5-one, and the addition salts thereof with an acid.

The coupler(s) may be present in said composition according to the invention in a concentration of between 0.0001% and 15% by weight relative to the total weight of the composition.

The dye composition in accordance with the invention may also contain at least one additional oxidation base other than the substituted para-phenylenediamine derivatives of formula (I) and/or at least one direct dye which differs from those of the present invention.

Among the additional oxidation bases that may be used according to the invention, mention may be made of para-phenylenediamine, para-tolylenediamine, 2-hydroxyethyl-para-phenylenediamine, 1-N,N-bis(2-hydroxyethyl)-para-phenylenediamine, para-aminophenols such as 3-methyl-4-aminophenol and 4-aminophenol, ortho-phenylenediamines, ortho-aminophenols, double bases, heterocyclic bases, for instance pyrimidines such as 2,4,5,6-tetraaminopyrimidine, or pyrazoles, such as 1-(2-hydroxyethyl)-4,5-diaminopyrazole.

The additional oxidation base(s) may be present in a concentration of between 0.0001% and 15% by weight relative to the total weight of said composition.

The medium for the composition that is suitable for dyeing is preferably an aqueous medium consisting of water and can advantageously contain cosmetically acceptable organic solvents including, more particularly, alcohols such as ethyl alcohol, isopropyl alcohol, benzyl alcohol and phenylethyl alcohol, or glycols or glycol ethers such as, for example, ethylene glycol monomethyl, monoethyl or monobutyl ether, propylene glycol or ethers thereof such as, for example, propylene glycol monomethyl ether, butylene glycol, dipropylene glycol and also diethylene glycol alkyl ethers such as, for example, diethylene glycol monoethyl ether or monobutyl ether in concentrations of between about 1% and about 40% and preferably between about 2% and 10% by weight relative to the total weight of the composition.

The composition according to the invention may also contain an effective amount of other agents that are previously known elsewhere in oxidation dyeing, such as various common adjuvants, for instance sequestering agents such as EDTA and etidronic acid, UV screening agents, waxes, volatile or nonvolatile, cyclic or linear or branched silicones, that are organomodified (especially with amine groups) or unmodified, preserving agents, ceramides, pseudoceramides, plant, mineral or synthetic oils, vitamins or provitamins, for instance panthenol, opacifiers, thickeners such as crosslinked polyacrylic acids or hydroxyalkylcelluloses, etc.

Said composition may also contain reducing agents or antioxidants. These may be chosen in particular from sodium sulfite, thioglycolic acid, thiolactic acid, sodium bisulfite, dehydroascorbic acid, hydroquinone, 2-methylhydroquinone, tert-butylhydroquinone and homogentisic acid, and they are then generally present in amounts ranging from about 0.05 to 1.5% by weight, relative to the total weight of the composition.

The composition according to the invention can also contain one or more fatty alcohols, these fatty alcohols being introduced in pure form or as a mixture. Among these, mention may be made more particularly of lauryl alcohol, cetyl alcohol, stearyl alcohol and oleyl alcohol and mixtures thereof. These fatty alcohols can represent from 0.001% to 20% by weight approximately relative to the total weight of the composition.

Preferably, the composition of the invention contains at least one nonionic, anionic, cationic or amphoteric surfactant in the proportion from about 0.1 to about 20% by weight.

Even more preferentially, said composition ontains at least one nonionic surfactant.

The composition according to the invention preferably contains at least one cationic or amphoteric substantive polymer in a proportion from about 0.05% to about 10% by weight.

Needless to say, a person skilled in the art will take care to select the optional additional compound(s) mentioned above, such that the advantageous properties intrinsically associated with the oxidation dye composition according to the invention are not, or are not substantially, adversely affected by the envisaged addition(s).

In the oxidizing composition, the oxidizing agent is preferably chosen from hydrogen peroxide, urea peroxide, alkali metal bromates or ferricyanides, and persalts such as perborates and persulfates. The use of hydrogen peroxide is particularly preferred. This oxidizing agent advantageously consists of an aqueous hydrogen peroxide solution whose titer can range more particularly from about 1 to about 40 volumes and even more preferentially from about 5 to about 40 volumes.

One or more redox enzymes such as 4-electron oxidoreductases such as laccases, peroxidases and 2-electron oxidoreductases (such as uricase), where appropriate in the presence of their respective donor or cofactor, may also be used as oxidizing agent.

The pH of the dye composition or of the composition ready to be used and applied to the keratin fibers [composition resulting from mixing the dye composition according to the invention and the oxidizing composition] is generally between the values 4 and 12. It is preferably between 6 and 11, and may be adjusted to the desired value by means of acidifying or basifying agents that are well known in the prior art in the dyeing of keratin fibers.

Among the basifying agents that may be mentioned, for example, are aqueous ammonia, alkali metal carbonates, alkanolamines such as monoethanolamine, diethanolamine and triethanolamine and also derivatives thereof, oxyethylenated and/or oxypropylenated hydroxyalkylamines and ethylenediamines, sodium hydroxide, potassium hydroxide and the compounds of formula (II) below:

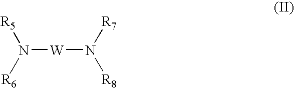

in which W is a propylene residue optionally substituted with a hydroxyl group or a C₁-C₄ alkyl radical; R₅, R₆, R₄ and R₈, which may be identical or different, represent a hydrogen atom, a C₁-C₄ alkyl radical or a C₁-C₄ hydroxyalkyl radical.

The acidifying agents are conventionally, for example, mineral or organic acids, for instance hydrochloric acid, orthophosphoric acid, carboxylic acids, for instance tartaric acid, citric acid or lactic acid, or sulfonic acids.

The dye composition in accordance with the invention may be in various forms, such as in the form of liquids, powders, creams or gels, that may be pressurized, or in any other form that is suitable for dyeing keratin fibers, and especially human hair.

The dyeing process according to the invention preferably consists in applying the ready-to-use composition, prepared extemporaneously at the time of use from the dye composition according to the invention and the oxidizing composition described above, to the wet or dry keratin fibers, and in leaving the composition to act for an exposure time preferably ranging from 1 to 60 minutes approximately and more preferably from 10 to 45 minutes approximately, rinsing the fibers and then optionally washing them with shampoo, followed by rinsing them again and drying them.

The examples that follow are intended to illustrate the invention.

### EXAMPLES

The dye compositions below, in accordance with the invention, were prepared:

| EXAMPLES | 1 | 2 |
|---|---|---|
| 1-(4′-Amino-3′-methylphenyl)-4-hydroxy-2- | 0.837 g | 0.837 g |
| methylpyrrolidine dihydrochloride | | |
| [substituted para-phenylenediamine | | |
| derivative of formula (I) in accordance | | |
| with the invention] | | |
| 2,4-Diamino-1-(β-hydroxyethyloxy) benzene | 0.723 g | 0.723 g |
| dihydrochioride | | |
| Alizarine | 0.15 g | |
| Basic Red 22 | | 0.2 g |
| Dye support (*) | qs | qs |
| Demineralized water qs | 100 g | 100 g |
| (*) Dye support | | |
| C₈-C₁₀ alkyl polyglucoside as an | | |
| aqueous 60% solution, sold under the | | |
| name Oramix CG lb ® by the company | | |
| SEPPIC | 3.24 | g AM* |
| Ethanol | 18.0 | g |
| Benzyl alcohol | 1.8 | g |
| Polyethylene glycol 400 | 2.7 | g |
| Pentasodium salt of diethylene- | | |
| triaminepentaacetic acid as an aqueous | | |
| 40% solution, sold under the name | | |
| Dissoluine D-40 ® by the company Akzo | 0.43 | g AM* |
| Sodium metabisulfite | 0.205 | g |
| Aqueous ammonia containing 20.5% NH₃ | 10.0 | g |

AM* denotes Active Material

At the time of use, the dye compositions described above were mixed weight for weight with a 20-volumes hydrogen peroxide solution (6% by weight).

The mixtures thus prepared were applied for 30 minutes to locks of permanent-waved natural gray hair containing 90% white hairs. The locks were then rinsed, washed with a standard shampoo, rinsed again and then dried.

The hair was dyed a strong violet-blue shade using composition 1 and a strong purple shade using composition 2.

1. A composition for the oxidation dyeing of keratin fibers, in particular of human keratin fibers such as the hair, characterized in that it comprises, in a medium that is suitable for dyeing:
(A) at least one oxidation base chosen from the substituted para-phenylenediamine derivatives of formula (I) below, and the addition salts thereof with an acid: 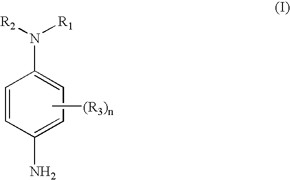
in which:
R₁ and R₂ can take one of the following meanings i) to v) below:
i) R₁ and R₂ simultaneously represent a —(CH₂)₂CHOHCH₂OH radical; or
ii) R₁ represents a —CH₂(CHOH)₄CH₂OH radical and R₂ represents a hydrogen atom, an alkyl or aryl radical or a heterocycle; or
iii) R₁ represents an alkyl or aryl radical or a heterocycle and R₂ represents an alkylene radical —(CH₂)ₘ— in which m is an integer equal to 2 or 3, said alkylene radical forming a ring together with the nitrogen atom, the carbon atom of the benzene ring bearing the nitrogen atom and one of the two carbon atoms of the benzene ring that are adjacent to it, it being understood that when R₁ is an alkyl or aryl radical, then either R₁ or said alkylene radical is substituted with a radical containing at least one nitrogen, oxygen or sulfur atom;
iv) R₁ represents a radical —(CH₂CH₂O)ₚR₄ in which p is an integer between 2 and 8 inclusive, R₄ and R₂, which may be identical or different, represent a hydrogen atom, an alkyl or aryl radical or a heterocycle;
v) R₁ and R₂ form, together with the nitrogen atom to which they are attached, a 5-, 6- or 7-membered saturated heterocycle, said heterocycle being substituted with at least one radical containing at least one carbon, nitrogen, oxygen or sulfur atom;
R₃ represents a halogen atom, an alkyl or aryl radical, a cyano, nitro, hydroxyl, carboxyl, sulfo, alkoxy, aryloxy, cyanoamino, amino, anilino, ureido, sulfamylamino, mono- or dialkylsulfamylamino, alkylthio, arylthio, alkoxycarbonylamino, sulfonamido, carbamyl, mono- or dialkylcarbamylsulfamyl, sulfonyl, alkoxycarbonyl, azo, acyloxy, carbamyloxy, mono- or dialkylcarbamyloxy, silyl, silyloxy, aryloxycarbonylamino, imido, sulfinyl, phosphonyl, aryloxycarbonyl, acyl or mercapto radical, a heterocycle or a heterocycle linked to the benzene ring of formula (I) via an ether or thio bond;
said alkyl radicals containing from 1 to 25 carbon atoms and possibly being linear, branched or cyclic and possibly being substituted with one or more radicals, and in this case possibly representing a mono- or polyhydroxyalkyl radical, an alkoxyalkyl radical, an aminoalkyl radical optionally substituted on the nitrogen atom, or a carboxyalkyl, alkylcarboxyalkyl, thioalkyl, alkylthioalkyl, cyanoalkyl, trifluoroalkyl, sulfoalkyl, phosphoalkyl or haloalkyl radical;
said alkoxy radicals containing from 1 to 25 carbon atoms and possibly being linear, branched or cyclic; said aryl radicals containing from 6 to 26 carbon atoms and possibly being substituted with one or more radicals chosen from alkyl, substituted alkyl and alkoxy radicals;
the heterocycles being monocyclic or polycyclic, each ring being 3-, 4-, 5- or 6-membered and possibly containing one or more hetero atoms, it being understood that in the case of polycyclic heterocycles, at least one of the rings contains at least one hetero atom such as N, O or S;
n is an integer between 0 and 4; it being understood that when n is greater than 1, then the radicals R₃ may be identical or different and may together form a 3-, 4-, 5- or 6-membered saturated or unsaturated ring; with the proviso that:
1) when R₁ and R₂ have the meanings defined in point v), then the compounds of formula (I) do not contain more than 3 hydroxyl radicals;
2) when R₁ and R₂ have the meanings defined in point v) and when R₁ and R₂ form a pyrrolidine ring substituted with a carbamoyl radical on the carbon in the alpha position to the nitrogen atom to which they are attached, then n is other than 0; or the pyrrolidine ring bears at least two substituents;
3) when R₁ and R₂ have the meanings defined in point v) and when R₁ and R₂ form a pyrrolidine ring substituted with a hydroxymethyl radical on the carbon in the alpha position relative to the nitrogen atom to which they are attached and when n=0 or 1, then either said ring bears at least two additional substituents, or said ring bears only one second substituent other than a hydroxyl radical on the carbon located in the β position relative to the nitrogen atom and relative to the carbon bearing said hydroxymethyl substituent; or alternatively when R₁ and R₂ have the meanings defined in point v) and when R₁ and R₂ form a pyrrolidine ring substituted with a hydroxymethyl radical on the carbon located in the alpha position relative to the nitrogen atom to which they are attached, and when n=1, then R₃ is other than an alkyl or mono- or polyhydroxyalkyl radical;
4) when R₁ and R₂ have the meanings defined in point iii), the compounds of formula (I) must satisfy at least one of the following four conditions:
a) irrespective of the value of n, the alkylene ring formed by the radical R₂ comprises a substituent in addition to the radical R₁; or
b) n is greater than 1; or
c) when n is equal to 1, then R₃ represents an aryl radical or a heterocycle; or
d) when n is equal to 0 or 1, then R₁ represents an aryl radical, a heterocycle or a substituted alkyl radical other than a monohydroxyalkyl radical;
5) R₁ and R₂ form a ring other than piperazine or diazacycloheptane;
(B) at least one synthetic direct dye chosen from azo dyes, quinone dyes, triarylmethane dyes, indoamine dyes or azine dyes and/or a natural dye.

2. The composition as claimed in claim 1, characterized in that, in formula (I), R₁ and R₂ take one of the meanings i) to v) below:
i) R₁ and R₂ simultaneously represent a —(CH₂)₂CHOHCH₂OH radical; or
ii) R₁ represents a —CH₂(CHOH)₄CH₂OH radical and R₂ represents a hydrogen atom or an alkyl radical; or
iv) R₁ represents a radical —(CH₂CH₂O)ₚR₄ in which p is an integer between 2 and 8 inclusive, R₄ and R₂, which may be identical or different, represent a hydrogen atom or an alkyl radical;
v) R₁ and R₂ form, together with the nitrogen atom to which they are attached, a 5-, 6- or 7-membered saturated heterocycle, said heterocycle being substituted with at least one radical containing at least one carbon, nitrogen or oxygen atom, which is not in a meta position relative to the nitrogen atom of the heterocycle;
R₃ represents a halogen atom, an alkyl or aryl radical or a heterocycle,
n is an integer equal to 0, 1 or 2.

3. The composition as claimed in claim 1 or 2, characterized in that, in formula (I), R₁ and R₂ form a pyrrolidine heterocycle.

4. The composition as claimed in claim 1 or 2, characterized in that the substituted para-phenylenediamine derivative(s) of formula (I) is(are) chosen from:
1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis-(3′,4′-dihydroxybutyl)-3-propyl-para-phenylenediamine, 1-N,N-bis(3′,4′-dihydroxybutyl)-3-methoxy-para-phenylenediamine, 1-N,N-bis(3′,4′-dihydroxybutyl)-3-ethoxy-para-phenylenediamine, 1-N,N-bis(3′,4′-dihydroxybutyl)-3-propyloxy-para-phenylenediamine, 1-N,N-bis(3′,4′-dihydroxybutyl)-3-hexyloxy-para-phenylenediamine, 1-N,N-bis(3′,4′-dihydroxybutyl)-3-(1″-N-3″,5″-dimethylpyrazolyl-para-phenylenediamine, 1-N,N-bis(3′,4′-dihydroxybutyl)-3-ureido-para-phenylenediamine, 1-N,N-bis(3′,4′-dihydroxybutyl)-3-trimethyl-1″,3″,3″-ureido-para-phenylenediamine, 1-N,N-bis(3′,4′-dihydroxybutyl)-3-dimethylamino-para-phenylenediamine, 1-N,N-bis(3′,4′-dihydroxybutyl)-3-methylthio-para-phenylenediamine, 1-N,N-bis(3′,4′-dihydroxybutyl)-3-ethylthio-para-phenylenediamine, 1-N,N-bis(3′,4′-dihydroxybutyl)-3-mercapto-para-phenylenediamine, 1-N,N-bis(3′,4′-dihydroxybutyl)-3-n-butylthio-para-phenylenediamine, 1-N,N-bis(3′,4′-dihydroxybutyl)-3-n-octylthio-para-phenylenediamine, 1-N,N-bis(3′,4′-dihydroxybutyl)-3-mercaptoethyl-para-phenylenediamine, 1-N,N-bis(3′,4′-dihydroxybutyl)-3-mercaptoethylthio-para-phenylenediamine, 1-N,N-bis(3′,4′-dihydroxybutyl)-3-β-hydroxyethylthio-para-phenylenediamine, 1-N-(2′,3′,4′,5′,6′-pentahydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(2′,3′,4′,5′,6′-pentahydroxyhexyl)-3-methoxy-para-phenylenediamine, 1-N-(2′,3′,4′,5′,6′-pentahydroxyhexyl)-1-N-(4″-N″-methylpiperidyl)-3-ethoxy-para-phenylenediamine, 1-N-(2′,3′,4′,5′,6′-pentahydroxyhexyl)-3-isopropyloxy-para-phenylenediamine, 1-N-(2′,3′,4′,5′,6′-pentahydroxyhexyl)-3-dimethylamino-para-phenylenediamine, 1-N-(2′,3′,4′,5′,6′-pentahydroxyhexyl)-3-methylthio-para-phenylenediamine, 1-N-(2′,3′,4′,5′,6′-pentahydroxyhexyl)-3-mercapto-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-isooctyloxy-para-phenylenediamine, 1-N-(methyl)-1-N-(2′,3′,4′,5′,6′-pentahydroxyhexyl)-3-isopropyloxy-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-3-hydroxyethyloxy-para-phenylenediamine, 1-N-(methyl)-1-N-(2′,3′,4′,5′,6′-pentahydroxyhexyl)-3-mercaptoethyloxy-para-phenylenediamine, 1-N-(methyl)-1-N-(2′,3′,4′,5′,6′-pentahydroxyhexyl)-para-phenylenediamine, 1-N-(phenyl)-1-N-(2′,3′,4′,5′,6′-pentahydroxyhexyl)-3-ethyloxy-para-phenylenediamine, -N-(4″-N-methylpiperidyl)-1-N-(2′,3′,4′,5′,6′-pentahydroxyhexyl)-3-ethyloxy-para-phenylenediamine, 4-N-(methyl)-4-N-(2′,3′,4′,5′,6′-pentahydroxyhexyl)-amino-7-amino-1-methylindole, 1-N-(hydroxyethyloxyethyl)-1-N-(2′,3′,4′,5′,6′-pentahydroxyhexyl)-3-ethyl-para-phenylenediamine, 1-N-(3′,4′-dihydroxybutyl)-5-aminoindoline, 1-(2′-hydroxyethyl)-2-methyl-5-aminoindoline, 1-methyl-2-hydroxymethyl-5-aminoindoline, 6-methyl-2-hydroxyethyl-5-aminoindoline, 2-hydroxyethyloxyethyl-5-aminoindoline, 2-hydroxyethyloxyethyloxyethyloxyethyl-5-aminoindoline, 2-hydroxyethyloxyethyloxyethyl-oxyethyloxyethyloxyethyl-6-isopropyl-5-aminoindoline, 2-hydroxyethyl-3-methyl-5-aminoindoline, 2-hydroxyethyloxyethyloxyethyl)-5-aminoindoline, 1-carboxymethyl-2,3,3-trimethyl-5-aminoindoline, 1-methylsulfonamidoethyl-3-methyl-5-aminoindoline, 1-ureidoethyl-6-methoxy-5-aminoindoline, 1-(2′,3′,4′,5′,6′-pentahydroxyhexyl)-5-aminoindoline, 1-N-(2′-mercaptoethyl)-5-aminoindoline, the dimethyl ester 6-amino-1-methyl-1,2,3,4-tetrahydrofuro-[2,3,h]-quinoline 4-methyl ester of phosphoric acid, 6-amino-1,2,2-trimethyl-4-trimethylsilanyloxy-1,2,3,4-tetrahydroquinoline, 6-amino-1-hexyl-2,2,7-trimethyl-4-mercaptomethyl-1,2,3,4-tetrahydroquinoline, 6-amino-1-(3′,4′-dihydroxybutyl)-2,2,3-trimethyl-1,2,3,4-tetrahydroquinoline, 6-amino-1-(ethoxyethoxyethoxyethoxy-3′,4′-dihydroxybutyl)-2,2,3,7-tetramethyl-1,2,3,4-tetrahydroquinoline, 6-amino-1-(hydroxyethyloxyethyloxyethyloxyethyl)-2,2,3-trimethyl-1,2,3,4-tetrahydroquinoline, 6-amino-1-(hydroxyethyloxyethyl)-2,2,3-trimethyl-1,2,3,4-tetrahydroquinoline, 6-amino-1-(ethylbis(hydroxyethyloxyethyloxyethyloxyethyl))-2,2,3,7-tetramethyl-1,2,3,4-tetrahydroquinoline, 1-(carboxymethyl)-2,2,3,7-tetramethyl-1,2,3,4-tetrahydroquinoline, 1-(hydroxypropyl)-2,2,3-trimethyl-7-methoxy-1,2,3,4-tetrahydroquinoline, 6-amino-1-(hydroxyethyloxyethyloxyethyl)-2,2,3-trimethyl-7-isopropyl-1,2,3,4-tetrahydroquinoline, 6-amino-1-(hydroxyethyloxyethyloxyethyloxyethyloxyethyloxyethyl-oxyethyloxyethyl)-2,2,3-trimethyl-7-isopropyl-1,2,3,4-tetrahydroquinoline, 6-amino-1-(hydroxyethyloxyethyloxyethyloxyethyloxyethyloxyethyl-oxyethyloxyethyl)-2,2,3-trimethyl-1,2,3,4-tetrahydroquinoline, 6-amino-1-(mercaptoethyl)-1,2,3,4-tetrahydroquinoline, 6-amino-1-(3′,4′-dihydroxybutyl)-2,2,3-trimethyl-7-isopropyl-1,2,3,4-tetrahydroquinoline, 6-amino-1-(3′,4′-dihydroxybutyl)-2,2,7-trimethyl-4-hydroxymethyl-1,2,3,4-tetrahydroquinoline, 6-amino-1-(3′,4′-dihydroxybutyl)-2,2-dimethyl-4-hydroxymethyl-7-isopropyl-1,2,3,4-tetrahydroquinoline, 6-amino-1-(3-hydroxypropyl)-2,2-dimethyl-4-hydroxymethyl-7-isopropyl-1,2,3,4-tetrahydroquinoline, 6-amino-1-(hydroxyethyloxyethyloxyethyloxyethyloxyethyloxy-ethyloxyethyloxyethyl)-2,2-dimethyl-4-hydroxymethyl-7-isopropyl-1,2,3,4-tetrahydroquinoline, 6-amino-1-(hydroxyethyloxyethyloxyethyloxyethyl)-2,2-dimethyl-4-hydroxymethyl-7-isopropyl-1,2,3,4-tetrahydroquinoline, 6-amino-1-(hydroxyethyloxyethyloxyethyloxyethyloxy-ethyl)-2,2-dimethyl-7-isopropyl-1,2,3,4-tetrahydroquinoline, 6-amino-1-(hydroxyethyloxyethyloxyethyloxyethyloxyethyloxyethyl)-2,2-dimethyl-7-isopropyl-1,2,3,4-tetrahydroquinoline, 6-amino-1-(hydroxyethyloxyethyl)-2,2-dimethyl-1,2,3,4-tetrahydroquinoline, 6-amino-1,2,2,4,7-pentamethyl-3-hydroxy-1,2,3,4-tetrahydroquinoline, 6-amino-1-(3′-hydroxypropyl)-4-(hydroxyethyloxyethyloxyethyloxy-ethyl)-2,2-dimethyl-7-isopropyl-1,2,3,4-tetrahydroquinoline, 6-amino-1-(hydroxyethyloxyethyloxyethyloxyethyl)-4,4-dimethyl-1,2,3,4-tetrahydroquinoline, 6-amino-1-(3′,4′-dihydroxybutyl)-2,2-dimethyl-7-isopropyl-1,2,3,4-tetrahydroquinoline, 6-amino-1-(hydroxyethyloxyethyloxyethyloxyethyl)-4-(hydroxyethyloxyethyloxyethyloxyethyl)-2,2,7-trimethyl-1,2,3,4-tetrahydroquinoline, 6-amino-1-(hydroxyethyloxyethyloxyethyloxyethyl)-2,2-dimethyl-7-isopropyl-1,2,3,4-tetrahydroquinoline, 6-amino-1-(2′,3′,4′,5′,6′-pentahydroxyhexyl)-2,2,4-trimethyl-7-isopropyl-1,2,3,4-tetrahydroquinoline, 6-amino-1-(mercaptoethyl)-2,2,4-trimethyl-7-(2′,3′-dihydroxypropyloxy)-1,2,3,4-tetrahydroquinoline, 6-amino-1-(3′,4′-dihydroxybutyl)-2,2,7-trimethyl-3-mercaptomethyl-1,2,3,4-tetrahydroquinoline, 6-amino-1-(ureidoethyl)-2,2,4-trimethyl-1,2,3,4-tetrahydroquinoline, 6-amino-2,2-dimethyl-7-chloro-1,2,3,4-tetrahydroquinoline-1-propylsulfonic acid, 6-amino-1-(4′-pyridinyl)-2,2,7-trimethyl-1,2,3,4-tetrahydroquinoline, 6-amino-1-(3′,4′-dihydroxybutyl)-2,2,4,7-tetramethyl-1,2,3,4-tetrahydroquinoline, 6-amino-1,7-diisopropyl-2,2-dimethyl-4-trimethylsilanyloxy-1,2,3,4-tetrahydroquinoline, 6-amino-1,2,2,4-tetramethyl-3-hydroxy-1,2,3,4-tetrahydroquinoline, 6-amino-1-bromo-2,2-dimethyl-4-mercapto-7-isopropyloxy-1,2,3,4-tetrahydroquinoline, 1-(4′-amino-3′-isopropyloxyphenyl)-2,6-dimethylpyrrolidine, 1-(4′-amino-3′-methylphenyl)-3-hydroxyethyloxypyrrolidine, 1-(4′-amino-3′-methylphenyl)-4-hydroxy-2-methylpyrrolidine, 1-(4′-amino-3′-methylphenyl)-3-methylsulfonamidopyrrolidine, 1-(4′-amino-3′-phenoxyphenyl)-3-methylsulfonamidopyrrolidine, 3-n-butylpyrrolidine-1-(4′-amino-3′-phenylsulfonic acid), 1-(4′-amino-3′-acetylaminophenyl)-3-hydroxymethylpyrrolidine, 7-amino-4-(2′-methyl)-pyrrolidinylbenzofuran, 1-(4′-aminophenyl)-2-(4″-aminophenoxymethyl)piperidine, 1-(4′-amino-3′-acetylphenyl)-4-hydroxypiperidine, 1-(4′-aminophenyl)-2-(hydroxyethyl)piperidine, 1-(4′-amino-3′-methoxyphenyl)-2,6-dihydroxymethylpiperidine, 1-(4′-amino-3′-isopropyloxyphenyl)-2,6-dimethylpiperidine, 1-(4′-amino-3′-isopropylphenyl)-2-hydroxymethylpiperidine, 1-(4′-amino-3′-isopropyloxyphenyl)-2-hydroxymethylpiperidine, 1-(4′-amino-3′-aminophenyl)-2-hydroxymethylpiperidine, 1-(4′-amino-3′-dimethylaminophenyl)-2-mercaptoethyloxyethylpiperidine, 1-(4′-amino-3′-(2″′,4″′-dichloro)anilinophenyl)-4-methylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-(4′-amino-3′-sulfamoylaminophenyl)-2,7-dimethylazacycloheptane, 1-(4′-amino-3′-methylthiophenyl)-2,7-dimethylazacycloheptane, 1-N-4′-hydroxybuty-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N-phenyl-1-N-(hydroxyethyloxyethyl)-para-phenylenediamine, 1-N-benzyl-1-N-(hydroxyethyloxyethyloxyethyloxyethyloxyethyl)-3-trimethylsilyl-para-phenylenediamine, 1-N-methyl-1-N-(hydroxyethyloxyethyloxyethyloxyethyloxyethyloxy-ethyloxyethyloxyethyl)-3-trimethylsilyloxy-para-phenylenediamine, 1-N-ethyl-1-N-(methoxyethyloxyethyloxyethyloxyethyloxyethyl)-3-phenoxycarbonylamino-para-phenylenediamine, 1-N-methyl-1-N-(methoxyethyloxyethyloxyethyl)-3-(2′,5′-dioxopyrrolidinyl)-para-phenylenediamine, 1-N-ethyl-1-N-(hydroxyethyloxyethyloxyethyl)-3-4′-pyridinylthio-para-phenylenediamine, 1-N-propyl-1-N-(hydroxyethyloxyethyloxyethyl)-3-sulfinyl-para-phenylenediamine, 1-N-methyl-1-N-(hydroxyethyloxyethyl)-3-phenoxycarbonyl-para-phenylenediamine, 1-N,N-bis(hydroxyethyloxyethyloxyethyloxyethyl)-para-phenylenediamine, 1-N,N-bis(methoxyethyloxyethyloxyethyloxyethyl)-3-isopropyloxy-para-phenylenediamine, 1-N,N-bis(hydroxyethyloxyethyloxyethyl)-3-isopropyloxy-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis(methoxyethyloxyethyloxyethyloxyethyloxyethyloxy-ethyloxyethyl)-3-methoxy-para-phenylenediamine, 1-N,N-bis(hydroxyethyloxyethyloxyethyloxyethyloxyethyloxy-ethyloxyethyloxyethyl)-3-methyl-para-phenylenediamine, 1-N,N-bis(hydroxyethyloxyethyloxyethyloxyethyl)-3-isopropyloxy-para-phenylenediamine, 1-N,N-bis(hydroxyethyloxyethyl)-3-mercaptoethyl-para-phenylenediamine and 1-N,N-bis(benzyloxyethyloxyethyloxyethyl)-3-isopropyl-para-phenylenediamine, and the addition salts thereof with an acid.

5. The composition as claimed in claim 4, characterized in that they are chosen from:
1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis(3′,4′-dihydroxybutyl)-3-propyl-para-phenylenediamine, 1-N-(2′,3′,4′,5′,6′-pentahydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-para-phenylenediamine and 1-N-(hydroxyethyloxyethyl)-1-N-(2′,3′,4′,5′,6′-pentahydroxyhexyl)-3-ethyl-para-phenylenediamine, and the addition salts thereof with an acid.

6. The composition as claimed in claim 4, characterized in that they are chosen from:
1-(4′-amino-3′-methylphenyl)-3-hydroxyethyloxypyrrolidine, 1-(4′-amino-3′-methylphenyl)-4-hydroxy-2-methylpyrrolidine, 1-(4′-amino-3′-methylphenyl)-3-methylsulfonamidopyrrolidine, 1-(4′-amino-3′-phenoxyphenyl)-3-methylsulfonamidopyrrolidine, 1-(4′-aminophenyl)-2-(4″-aminophenoxymethyl)piperidine, 1-(4′-aminophenyl)-2-hydroxyethyl)piperidine, 1-(4′-amino-3′-isopropylphenyl)-2-hydroxymethylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-N-4′-hydroxybutyl-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N,N-bis(hydroxyethyloxyethyloxyethyloxyethyl)-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyloxyethyloxy-ethyloxyethyloxyethyl)-3-methyl-para-phenylenediamine and 1-N,N-bis(benzyloxyethyloxyethyloxyethyl)-3-isopropyl-para-phenylenediamine.

7. The composition as claimed in any one of the preceding claims, characterized in that the addition salts with an acid are chosen from the hydrochlorides, hydrobromides, sulfates, tartrates, lactates and acetates.

8. The composition as claimed in any one of the preceding claims, characterized in that the substituted para-phenylenediamine derivative(s) of formula (I) and/or the addition salt(s) thereof with an acid represent from 0.0001% to 20% by weight relative to the total weight of the composition.

9. The composition as claimed in claim 8, characterized in that the substituted para-phenylenediamine derivative(s) of formula (I) and/or the addition salt(s) thereof with an acid represent from 0.001% to 15% by weight relative to the total weight of the composition.

10. The composition as claimed in claim 9, characterized in that the substituted para-phenylenediamine derivative(s) of formula (I) and/or the addition salt(s) thereof with an acid represent from 0.01% to 10% by weight relative to the total weight of the composition.

11. The composition as claimed in any one of the preceding claims, characterized in that the synthetic azo direct dye(s) is(are) nonionic, anionic or cationic.

12. The composition as claimed in claim 11, characterized in that the synthetic azo direct dye(s) is (are) chosen from—Disperse Red 17—Acid Yellow 9—Acid Black 1—Basic Red 22—Basic Red 76—Basic Yellow 57—Basic Brown 16—Acid Yellow 36—Acid Orange 7—Acid Red 33—Acid Red 35—Basic Brown 17—Acid Yellow 23—Acid Orange 24—Disperse Black 9,—1-(4′-(aminodiphenylazo)-2-methyl-4-bis(β-hydroxy-ethyl)aminobenzene and—4-hydroxy-3-(2-methoxyphenylazo)-1-naphthalenesulfonic acid.

13. The composition as claimed in any one of claims 1 to 10, characterized in that the synthetic quinone direct dye(s) is(are) chosen from—Disperse Red 15—Solvent Violet 13—Acid Violet 43—Disperse Violet 1—Disperse Violet 4—Disperse Blue 1—Disperse Violet 8—Disperse Blue 3—Disperse Red 11—Acid Blue 62—Disperse Blue 7—Basic Blue 22—Disperse Violet 15—Basic Blue 99,—1-N-methylmorpholiniumpropylamino-4-hydroxy-anthraquinone,—1-aminopropylamino-4-methylaminoanthraquinone,—1-aminopropylaminoanthraquinone,—5-β-hydroxyethyl-1,4-diaminoanthraquinone,—2-aminoethylaminoanthraquinone,—1,4-bis(β,γ-dihydroxypropylamino)anthraquinone.

14. The composition as claimed in any one of claims 1 to 10, characterized in that the synthetic azine direct dye(s) is (are) chosen from Basic Blue 17 and Basic Red 2.

15. The composition as claimed in any one of claims 1 to 10, characterized in that the synthetic triarylmethane direct dye(s) is(are) chosen from—Basic Green 1—Acid Blue 9—Basic Violet 3—Basic Violet 14—Basic Blue 7—Acid Violet 49—Basic Blue 26—Acid Blue 7.

16. The composition as claimed in any one of claims 1 to 10, characterized in that the synthetic indoamine direct dye(s) is(are) chosen from 2-β-hydroxyethylamino-5-[bis(β-4′-hydroxyethyl)-amino]anilino-1,4-benzoquinone, 2-β-hydroxyethylamino-5-(2′-methoxy-4′-amino)-anilino-1,4-benzoquinone, 3-N-(2′-chloro-4′-hydroxy)phenylacetylamino-6-methoxy-1,4-benzoquinoneimine, 3-N-(3′-chloro-4′-methylamino)-phenylureido-6-methyl-1,4-benzoquinoneimine, 3-[4′-N-(ethylcarbamylmethyl)amino]phenylureido-6-methyl-1,4-benzoquinoneimine.

17. The composition as claimed in any one of claims 1 to 10, characterized in that the natural dye(s) is(are) chosen from lawsone, juglone, alizarin, purpurin, carminic acid, kermesic acid, purpurogallin, protocatechaldehyde, indigo, isatin, curcumin, spinulosin, apigenidin, extracts or decoctions containing these natural dyes and especially henna-based poultices or extracts.

18. The composition as claimed in any one of the preceding claims, characterized in that the synthetic direct dye(s) according to the invention and/or the natural dye(s) represent from 0.001% to 20% by weight relative to the total weight of the composition.

19. The composition as claimed in claim 18, characterized in that the synthetic direct dye(s) according to the invention and/or the natural dye(s) represent from 0.005% to 10% by weight relative to the total weight of the composition.

20. The composition as claimed in any one of the preceding claims, characterized in that it contains at least one coupler.

21. The composition as claimed in claim 20, characterized in that the coupler(s) is (are) chosen from meta-phenylenediamines, meta-aminophenols, meta-diphenols and heterocyclic couplers, such as, for example, indole derivatives, indoline derivatives, benzimidazole derivatives, benzomorpholine derivatives, sesamol derivatives and pyridine, pyrimidine and pyrazole derivatives, and the addition salts thereof with an acid.

22. The composition as claimed in claim 20 or 21, characterized in that the coupler(s) is(are) chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 2-methyl-1-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one and 1-phenyl-3-methylpyrazol-5-one, and the addition salts thereof with an acid.

23. The composition as claimed in any one of claims 20 to 22, characterized in that the coupler(s) is (are) present in a concentration of between 0.0001% and 15% by weight relative to the total weight of the composition.

24. The composition as claimed in any one of the preceding claims, characterized in that it contains at least one additional oxidation base.

25. The composition as claimed in claim 24, characterized in that the additional oxidation base(s) is (are) chosen from para-phenylenediamine, para-tolylenediamine, 2-hydroxyethyl-para-phenylenediamine, 1-N,N-bis(2-hydroxyethyl)-para-phenylenediamine, para-aminophenols, ortho-phenylenediamines, ortho-aminophenols, double bases and heterocyclic bases.

26. The composition as claimed in claims 24 and 25, characterized in that the additional oxidation base(s) is(are) present in a concentration of between 0.0001% and 15% by weight relative to the total weight of the composition.

27. The composition as claimed in any one of the preceding claims, characterized in that the medium that is suitable for dyeing consists of water or a mixture of water and at least one organic solvent.

28. The composition as claimed in claim 27, characterized in that the solvents are present in proportions of between 1% and 40% by weight relative to the total weight of the composition.

29. The composition as claimed in any one of the preceding claims, characterized in that it contains sequestering agents, UV screening agents, waxes, volatile or nonvolatile, cyclic or linear or branched silicones, that may be organomodified (particularly by amine groups) or unmodified, preserving agents, ceramides, pseudoceramides, plant, mineral or synthetic oils, vitamins or provitamins, opacifiers, thickeners, reducing agents or antibxidants, fatty alcohols and acidifying or basifying agents.

30. The composition as claimed in any one of the preceding claims, characterized in that it contains at least one anionic, nonionic, cationic or amphoteric surfactant and preferably a nonionic surfactant in a proportion ranging from 0.1% to 20% by weight.

31. The composition as claimed in any one of the preceding claims, characterized in that it contains at least one cationic or amphoteric polymer in a proportion ranging from 0.05% to 10% by weight.

32. A ready-to-use composition for the oxidation dyeing of keratin fibers, in particular of human keratin fibers such as the hair, characterized in that it is obtained by mixing a composition as defined in any one of claims 1 to 31 and a composition containing at least one oxidizing agent.

33. The composition as claimed in claim 32, characterized in that the pH is between the values 4 and 12 and preferably is between 6 and 11.

34. The composition as claimed in any one of the preceding claims, characterized in that it is in the form of liquids, powders, creams or gels, that are optionally pressurized, or in any other form that is suitable for dyeing keratin fibers, and especially human hair.

35. The composition as claimed in claim 32, characterized in that the oxidizing agent is chosen from hydrogen peroxide, urea peroxide, alkali metal romates or ferricyanides, persalts such as perborates and persulfates, and redox enzymes such as laccases, peroxidases and 2-electron oxidoreductases, where appropriate in the presence of their respective donor or cofactor.

36. A composition as claimed in claim 35, characterized in that the oxidizing agent comprises an aqueous hydrogen peroxide solution with a titer ranging from 1 to 40 volumes and preferably 5 to 40 volumes.

37. A process for the oxidation dyeing of keratin fibers, and in particular of human keratin fibers such as the hair, which consists in applying to the fibers a composition which consists in applying to the fibers a dye composition containing, in a medium that is suitable for dyeing, at least one substituted para-phenylenediamine derivative of formula (I) defined in any one of claims 1 to 10, in combination with at least one synthetic azo, quinone, triarylmethane, indoamine or azine direct dye and/or a natural dye defined in any one of claims 11 to 17, the color being developed at alkaline, neutral or acidic pH with the aid of a composition containing at least one oxidizing agent, which is mixed just at the time of use to the dye composition, or which is applied sequentially without intermediate rinsing.

38. The process as claimed in claim 37, characterized in that it consists in applying the ready-to-use composition, prepared extemporaneously at the time of use from the composition according to the invention and the oxidizing composition, to the wet or dry keratin fibers,-and leaving it to act for an exposure time preferably ranging from 1 to 60 minutes approximately and more preferably from 10 to 45 minutes approximately, rinsing the fibers and then optionally washing them with shampoo, followed by rinsing them again and drying them.

39. A multi-compartment device or “kit” for the oxidation dyeing of keratin fibers, in particular human keratin fibers such as the hair, which comprises at least one compartment containing at least one substituted para-phenylenediamine derivative of formula (I) defined according to any one of claims 1 to 10 and at least one synthetic azo, quinone, triarylmethane, indoamine or azine direct dye and/or a natural dye, and at least one other compartment containing at least one oxidizing agent.

40. A multi-compartment device or “kit” for the oxidation dyeing of keratin fibers, in particular human keratin fibers such as the hair, which comprises at least one compartment containing at least one substituted para-phenylenediamine derivative of formula (I) defined according to any one of claims 1 to 10, at least one compartment containing at least one synthetic azo, quinone, triarylmethane, indoamine or azine direct dye and/or a natural dye and at least one other compartment containing at least one oxidizing agent.
